(19) United States
(12) Patent Application Publication
(10) Pub. No.: US 2013/0225595 A1
(43) Pub. Date: Aug. 29, 2013

(54) METHOD FOR TREATING PULMONARY ARTERIAL HYPERTENSION IN A PATIENT NOT HAVING IDIOPATHIC PULMONARY FIBROSIS

(71) Applicant: Gilead Sciences, Inc., (US)

(72) Inventors: Hunter Campbell Gillies, El Granada, CA (US); Tobias Peschel, Redwood City, CA (US); David J. Pizzuti, Menlo Park, CA (US)

(73) Assignee: Gilead Sciences, Inc., Foster City, CA (US)

(21) Appl. No.: 13/830,211

(22) Filed: Mar. 14, 2013

Related U.S. Application Data
(63) Continuation-in-part of application No. 13/571,039, filed on Aug. 9, 2012.
Application No. 13/571,039 is a continuation of application No. 13/536,001, filed on Jun. 28, 2012.
(60) Provisional application No. 61/605,002, filed on Feb. 29, 2012.

(51) Int. Cl. A61K 31/505; A61K 45/06

(52) U.S. Cl. CPC A61K 31/505; A61K 45/06
USPC 514/250; 514/274

(57) ABSTRACT

There is provided a method of treating pulmonary hypertension in a patient in need thereof, said method comprising: administering a therapeutically effective amount of ambrisentan to the patient with pulmonary arterial hypertension, wherein the patient has been determined not to have idiopathic pulmonary fibrosis.

### CROSS-REFERENCE TO RELATED APPLICATION

This is a continuation-in-part application of U.S. application Ser. No. 13/571,039 filed Aug. 9, 2012 which is a continuation application of U.S. application Ser. No. 13/536,001 filed Jun. 28, 2012, which claims the benefit and priority to U.S. Provisional Application No. 61/605,002 filed Feb. 29, 2012. The entire disclosure of the applications identified in this paragraph is incorporated herein by references.

### FIELD

The present disclosure relates to methods useful for treating a subject having a pulmonary hypertension condition, and for improving clinical outcome in such a subject. Particularly, the present disclosure relates to methods for treating a pulmonary hypertension condition in a subject who does not have idiopathic pulmonary fibrosis.

### BACKGROUND

This section provides background information related to the present disclosure which is not necessarily prior art.

Pulmonary hypertension (PH) has been previously classified as primary (idiopathic) or secondary. Recently, the World Health Organization (WHO) has classified pulmonary hypertension into five groups:
Group 1: pulmonary arterial hypertension (PAH);
Group 2: PH with left heart disease;
Group 3: PH with lung disease and/or hypoxemia;
Group 4: PH due to chronic thrombotic and/or embolic disease; and
Group 5: miscellaneous conditions (e.g., sarcoidosis, histiocytosis X, lymphangiomatosis and compression of pulmonary vessels).

Pulmonary arterial hypertension (PAH) is a serious, progressive and life-threatening disease of the pulmonary vasculature, characterized by profound vasoconstriction and an abnormal proliferation of smooth muscle cells in the walls of the pulmonary arteries. Severe constriction of the blood vessels in the lungs leads to very high pulmonary arterial pressures. These high pressures make it difficult for the heart to pump blood through the lungs to be oxygenated. Patients with PAH suffer from extreme shortness of breath as the heart struggles to pump against these high pressures. Patients with PAH typically develop significant increases in pulmonary vascular resistance (PVR) and sustained elevations in pulmonary artery pressure (PAP), which ultimately lead to right ventricular failure and death. Patients diagnosed with PAH have a poor prognosis and equally compromised quality of life, with a mean life expectancy of 2 to 5 years from the time of diagnosis if untreated.

Endothelin-1 (ET-1) is the primary member of a family of potent vasoconstrictor peptides, which are known to play an essential role in mammalian cardiovascular physiology. ET-1 is synthesized de novo and released from endothelial cells in response to a variety of factors, including angiotensin II, catecholamines, cytokines, hypoxia and shear stress. Two receptor subtypes, endothelin receptor type A (ET_{A}) and endothelin receptor type B (ET_{B}), mediate the effects of ET-1. In humans, the ET_{A} receptor is preferentially expressed in vascular smooth muscle cells and is primarily responsible for the vasoconstrictive effects of ET-1. In contrast, ET_{B} receptors are found mainly in the vascular endothelium, and their activation results in vasodilatation via production of nitric oxide and prostacyclin. The ET_{B} receptor is also involved in regulation of circulating concentrations of ET-1, through effects on endothelin converting enzyme (ECE-1) expression, and the synthesis and reuptake of ET-1 by endothelial cells.

Ambrisentan is a non-sulfonamide, propanoic acid-class endothelin receptor antagonist (ERA) with high affinity (about 12 pM) for the ET_{A} receptor. Bosentan, a non-selective, sulfonamide-class ERA, is approved for treatment of PAH in patients with WHO functional class II to IV symptoms. Sitaxsentan is another sulfonamide-class ERA that is selective for the ET_{A} receptor. Pfizer voluntarily removed sitaxentan from the market due to concerns about liver toxicity (see Pfizer News Release dated Dec. 10, 2010, http://pfizer.mediaroom.com/index.php?s=5149&item=22387|˜http://pfizer.mediaroom.com/index.php?s=5149&item=22387%0A %09%09%09%09).

Myogen, Inc. News Release dated May 19, 2005 reports initiation of a clinical trial to evaluate ambrisentan in patients with PAH who have previously discontinued bosentan or sitaxsentan therapy due to liver function test (LFT) abnormalities, specifically elevated serum aminotransferase concentrations.

U.S. Patent Application Publication No. 2008/0139593 mentions that both the 5 mg and 10 mg dose of ambrisentan administered once daily provided statistically significant and clinically relevant improvements in exercise capacity and symptoms in subjects with PAH. U.S. 2008/0139593 also states that serum aminotransferase abnormalities, which have been observed and treatment-limiting for other ERAs, were not observed in any subjects receiving ambrisentan.

Hartmann, et al., Can. J. Physiol. Pharmacol. 88, 682-691 (2010), using human hepatocytes to compare effects of endothelin receptor antagonists on hepatobiliary transport, reports that bosentan and sitaxsentan decreased transporter activity to the greatest extent, while ambrisentan and darusentan were less potent. Hartmann also states that clinically, ambrisentan is associated with a lower incidence of serum aminotransferase elevations than bosentan and sitaxsentan and the present results begin to provide a mechanistic explanation for this difference.

In summarizing post-marketing data on PAH patients treated with ambrisentan, McGoon, et al. (Poster 1061, presented at 9^{th} Int. PH Conference, June 2010) report that the totality of data does not support an association of ambrisentan with increased risk of drug induced hepatotoxicity.

The FDA approved Prescribing Label for ambrisentan (LETAIRIS®), revised June 2007, provides a black box warning that states “Elevations of liver aminotransferases (ALT, AST) have been reported with LETAIRIS® and serious liver injury has been reported with related drugs.” The label further states that liver chemistries must be measured prior to initiation of LETAIRIS® and at least every month thereafter. The label also requires that LETAIRIS® be obtained through a special restricted distribution program involving patient registration.

Kingman, et al., Expert Opin. Pharmacother. 10, 1847-1858 (2009) report that significant aminotransferase abnormalities were not observed with ambrisentan treatment in the placebo-controlled trials, and in all clinical trials combined the 1-year risk seems to be low (<3%). However, Kingman mentions that despite these data, the FDA requires monthly liver function test monitoring.

Any promotional activity which is inconsistent with the black box warning may constitute misbranding of a drug product which is a violation of the Federal Food, Drug, and Cosmetic Act, 21 U.S.C. 352(f)(1) and (n). On Feb. 27, 2009, the FDA sent Gilead (the NDA sponsor of LETAIRIS®) a letter to notify that the agency came across some statements made by a Gilead representative that might have violated the Act by minimizing potentially serious risks associated with LETAIRIS®. Gilead explained in its response dated Mar. 6, 2009 that there was misunderstanding by the FDA and all marketing activities of Gilead were compliant with the FDA guideline. After further review, the FDA acknowledged on Mar. 27, 2009 that Gilead “has reviewed all Letairis materials in use and has concluded that they are compliant with each applicable requirement of the Act and FDA implementing regulations” and closed the case on compliance investigation.

Antoniu, Expert Opin Ther Targets 2008 Sep. 1; 12(9):1077-84 reports that bosentan, a non-selective ETA antagonist, is a promising anti-fibrotic therapy for IPF and clinical data on its long-term efficacy support its use.

Knobloch et al, 2010 Annual Congress of the European Respiratory Society (ERS) (September 2010) mentions that an ET-receptor antagonist has therapeutic utility for early stage inflammation/fibrosis-associated chronic airway diseases by counteracting the establishment of inflammatory and fibrotic processes.”

Henderson et al, 2010 Annual Congress of the European Respiratory Society (ERS) (September 2010) suggests that a selective ETA antagonist such as ambrisentan has potential utility as a novel therapeutic agent for pulmonary fibrosis.

Gilead Sciences News Release dated Dec. 22, 2010 announces that it is stopping ARTEMIS-IPF, the company's ongoing Phase III clinical trial of ambrisentan in patients with idiopathic pulmonary fibrosis (IPF), due to lack of efficacy.

### SUMMARY

This section provides a general summary of the invention and is not a comprehensive disclosure of all of its features.

As described above in the background, there are earlier reports suggesting that a selective ETA antagonist has potential therapeutic utility for the treatment of pulmonary fibrosis. However, it is surprisingly discovered that ambrisentan could cause a greater risk of idiopathic pulmonary fibrosis progression. In an embodiment, therefore, there is provided a method of treating pulmonary hypertension in a patient in need thereof, said method comprising: administering a therapeutically effective amount of ambrisentan to the patient with pulmonary arterial hypertension, wherein the patient has been determined not to have idiopathic pulmonary fibrosis.

In another embodiment, there is provided a method of treating arterial pulmonary hypertension in a patient in need thereof, comprising: administering a therapeutically effective amount of ambrisentan to the patient with pulmonary arterial hypertension; wherein the patient has been determined not to have idiopathic pulmonary fibrosis, and wherein the method is carried out without drug labeling instruction to monitor one or more biomarkers of liver function during ambrisentan treatment.

In yet another embodiment, there is provided a method of treating pulmonary arterial hypertension in a patient in need thereof, comprising: diagnosing a patient with pulmonary arterial hypertension; screening the patient for idiopathic pulmonary fibrosis; determining that the patient does not have idiopathic pulmonary fibrosis; and administering a therapeutically effective amount of ambrisentan to the patient with pulmonary arterial hypertension.

### BRIEF DESCRIPTION OF THE DRAWINGS

FIG. 1 shows evaluation of drug-induced serious hepatotoxicity (eDISH) plot comparing maximum bilirubin values to maximum alanine aminotransferase (ALT).

FIG. 2 shows eDISH plot comparing maximum bilirubin values to maximum aspartate aminotransferase (AST).

FIG. 3 shows Letairis Education and Access Program (LEAP) patients by exposure duration.

FIG. 4 shows empiric Bayesian geometric mean (EBGM) scores for ambrisentan and bosentan hepatic events.

FIG. 5 is a chart showing mean change in 6-minute walk distance in the placebo and ambrisentan groups.

FIG. 6 is a chart showing time to clinical worsening with Kaplan-Meier estimates of the proportions of failures in ARIES-1 and ARIES-2.

### DETAILED DESCRIPTION

### Definitions

As used in the present specification, the following words and phrases are generally intended to have the meanings as set forth below, except to the extent that the context in which they are used indicates otherwise.

“Ambrisentan” or “AMB” is described in U.S. Pat. Nos. 5,703,017; 5,932,730 and 7,109,205. It refers to the chemical compound, (25)-2-[(4,6-dimethylpyrimidin-2-yl)oxy]-3-methoxy-3,3-diphenylpropanoic acid and has the following chemical formula:

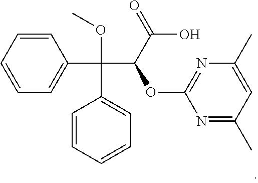 [SMILES: COC(c1ccccc1)(c1ccccc1)[C@H](Oc1nc(C)cc(C)n1)C(=O)O]

Ambrisentan is approved for sale by the U.S. Food and Drug Administration (FDA) for once-daily treatment of PAH and is marketed under the trade name Letairis®. In Europe, Ambrisentan is approved under the trade name Volibris®.

“Ambrisentan” as used herein is intended to include the metabolites of ambrisentan described in U.S. Patent Publication No. 2010/0204163. The ambrisentan metabolites include the compounds of the following chemical Formula (I):

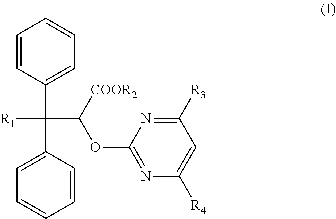

wherein R¹ is —OH or —OCH₃; R² is —H, lower alkyl or glycosidyl; and R³ and R⁴ are independently —CH₃, —C(O)H or —CH₂OR⁶, wherein R⁶ is —H or a hydrocarbyl group having 1 to 20 carbon atoms.

“Ambrisentan” as used herein is also intended to include a pharmaceutically acceptable salt thereof. The term “salt” refers to a pharmaceutically acceptable salt of a compound that includes a variety of physiologically acceptable organic and inorganic counter ions known to one of skill in the art.

The term, “companion diagnosis”, refers to a diagnosis designed to provide information that is essential for the safe and effective use of a corresponding therapeutic product, for example, ERAs. Such a companion diagnosis can identify appropriate subpopulations for treatment or identify populations who should not receive a particular treatment because of an increased risk of a serious side effect such as hepatotoxicity caused by ERA's. In various embodiments, the companion diagnosis is intended to monitor liver aminotransferase levels.

The term “monitor” or “monitoring” refers to an evaluation of a disease or condition over time. It can be performed by continuously and/or repeatedly measuring certain parameters, for example, by continuously or repeatedly performing medical tests such as, for example, measurement of liver aminotransferase levels.

The term “treatment” or “treating” means any administration of a drug to a subject, such as a mammal according to the method of the invention for purposes including: 1) preventing or protecting against a disease or condition, that is, causing the clinical symptoms not to develop; 2) inhibiting the disease or condition, that is, arresting or suppressing the development of clinical symptoms; and/or 3) relieving the disease or condition that is, causing the regression of clinical symptoms.

“Oral administration” is a route of administration where a substance is taken through the mouth, and includes buccal, sublabial and sublingual administration, as well as enteral administration and through the respiratory tract, unless made through, e.g., tubing so the medication is not in direct contact with any of the oral mucosa. Typical form for the oral administration of therapeutic agents includes the use of tablets or capsules.

### Pulmonary Hypertension and Treatment Thereof

The present disclosure provides a method of treating pulmonary hypertension in a patient in need thereof, said method comprising: administering a therapeutically effective amount of ambrisentan to the patient with pulmonary arterial hypertension, wherein the patient has been determined not to have idiopathic pulmonary fibrosis. In another embodiment, there is provided a method of treating arterial pulmonary hypertension in a patient in need thereof, comprising: administering a therapeutically effective amount of ambrisentan to the patient with pulmonary arterial hypertension; wherein the patient has been determined not to have idiopathic pulmonary fibrosis, and wherein the method is carried out without drug labeling instruction to monitor one or more biomarkers of liver function during ambrisentan treatment. In yet another embodiment, there is provided a method of treating pulmonary arterial hypertension in a patient in need thereof, comprising: diagnosing a patient with pulmonary arterial hypertension; screening the patient for idiopathic pulmonary fibrosis; determining that the patient does not have idiopathic pulmonary fibrosis; and administering a therapeutically effective amount of ambrisentan to the patient with pulmonary arterial hypertension.

Diagnosis of a pulmonary hypertension condition is confirmed hemodynamically, for example in the case of PAH by presence of one or more, and more typically two or all three of the following (McLaughlin et al. JACC Vol. 53, No. 17, p 1573-1619 (2009)):
(a) mean pulmonary arterial pressure (PAP) not less than about 25 mmHg at rest;
(b) pulmonary vascular resistance (PVR) not less than about 3 mmHg/liter/minute;
(c) pulmonary capillary wedge pressure (PCWP) or left ventricle end diastolic pressure (LVEDP) not greater than about 15 mmHg.

The pulmonary hypertension condition diagnosed, and treated by a method of this disclosure, can comprise any one or more of the conditions recognized according to the current World Health Organization (WHO) classification (see, for example, Simonneau (2009) JACC 54:S43-54):

1. Pulmonary arterial hypertension (PAH)

1.1. Idiopathic PAH

1.2. Heritable
1.2.1. BMPR2
1.2.2. ALK1, endoglin (with or without hereditary hemorrhagic telangiectasia)
1.2.3. Unknown

1.3. Drug- and toxin-induced

1.4. Associated with
1.4.1. Connective tissue diseases
1.4.2. HIV infection
1.4.3. Portal hypertension
1.4.4. Congenital heart diseases
1.4.5. Schistosomiasis
1.4.6. Chronic hemolytic anemia

1.5 Persistent pulmonary hypertension of the newborn

1′. Pulmonary veno-occlusive disease (PVOD) and/or pulmonary capillary hemangiomatosis (PCH)
2. Pulmonary hypertension owing to left heart disease

2.1. Systolic dysfunction

2.2. Diastolic dysfunction

2.3. Valvular disease

3. Pulmonary hypertension owing to lung diseases and/or hypoxia

3.1. Chronic obstructive pulmonary disease

3.2. Interstitial lung disease

3.3. Other pulmonary diseases with mixed restrictive and obstructive pattern

3.4. Sleep-disordered breathing

3.5. Alveolar hypoventilation disorders

3.6. Chronic exposure to high altitude

3.7. Developmental abnormalities

4. Chronic thromboembolic pulmonary hypertension (CTEPH)
5. Pulmonary hypertension with unclear multifactorial mechanisms

5.1. Hematologic disorders: myeloproliferative disorders, splenectomy

5.2. Systemic disorders: sarcoidosis, pulmonary Langerhans cell histiocytosis: lymphangioleiomyomatosis, neurofibromatosis, vasculitis

5.3. Metabolic disorders: glycogen storage disease, Gaucher disease, thyroid disorders

5.4. Others: tumoral obstruction, tibrosing mediastinitis, chronic renal failure on dialysis

In one embodiment, the pulmonary hypertension condition comprises PAH (WHO Group 1), for example idiopathic PAH, heritable PAH or PAH associated with another disease or condition. Pulmonary hypertension at baseline can be mild, moderate or severe, as measured for example by WHO functional class, which is a measure of disease severity in patients with pulmonary hypertension. The WHO functional classification is an adaptation of the New York Heart Association (NYHA) system and is routinely used to qualitatively assess activity tolerance, for example in monitoring disease progression and response to treatment (Rubin (2004) Chest 126:7-10). Four functional classes are recognized in the WHO system:
Class I: pulmonary hypertension without resulting limitation of physical activity; ordinary physical activity does not cause undue dyspnea or fatigue, chest pain or near syncope;
Class II: pulmonary hypertension resulting in slight limitation of physical activity; patient comfortable at rest; ordinary physical activity causes undue dyspnea or fatigue, chest pain or near syncope;
Class III: pulmonary hypertension resulting in marked limitation of physical activity; patient comfortable at rest; less than ordinary activity causes undue dyspnea or fatigue, chest pain or near syncope;
Class IV: pulmonary hypertension resulting in inability to carry out any physical activity without symptoms; patient manifests signs of right-heart failure; dyspnea and/or fatigue may be present even at rest; discomfort is increased by any physical activity.

In an embodiment, the subject at baseline exhibits pulmonary hypertension (e.g., PAH) of at least WHO Class II, for example WHO Class II or Class III, but does not have idiopathic pulmonary fibrosis (IPF).

In another embodiment, the subject at baseline exhibits mean PAP at rest of at least about 30 mmHg, for example at least about 35, at least about 40, at least about 45 or at least about 50 mmHg. The term, “baseline”, herein means a time immediately prior to initiation of treatment with ambrisentan.

Treatment of pulmonary hypertension encompasses one or more of the following:
(a) adjustment of one or more hemodynamic parameters towards a more normal level, for example lowering mean PAP or PVR, PCWP or LVEDP, or raising cardiac index or output versus baseline;
(b) improvement of cardiopulmonary efficiency versus baseline, for example increasing exercise capacity, illustratively as measured in a test of 6-minute walking distance (6MWD), or lowering Borg dyspnea index (BDI);
(c) improvement of one or more quality of life parameters versus baseline, for example an increase in score on at least one of the SF-36® health survey functional scales;
(d) general improvement versus baseline in the severity of the condition, for example by movement to a lower WHO functional class;
(e) improvement of clinical outcome following a period of treatment, versus expectation in absence of treatment (e.g., in a clinical trial setting, as measured by comparison with placebo), including improved prognosis, extending time to or lowering probability of clinical worsening, extending quality of life (e.g., delaying progression to a higher WHO functional class or slowing decline in one or more quality of life parameters such as SF-36® health survey parameters), and/or increasing longevity; and/or
(f) adjustment towards a more normal level of one or more molecular markers that can be predictive of clinical outcome (e.g., plasma concentrations of endothelin-1 (ET-1), cardiac troponin T (cTnT) or B-type natriuretic peptide (BNP)).

A therapeutically effective amount of ambrisentan is an amount (typically a daily amount administered over the course of a period of treatment) sufficient to provide any one or more of the effects mentioned above. Typically, a therapeutically effective amount will be found in the range of about 1 to about 15 mg/day, for example about 2 to about 15 mg/day, about 2.5 to about 10 mg/day, or about 2.5, about 3, about 3.5, about 4, about 4.5, about 5, about 6, about 7, about 8, about 9 or about 10 mg/day.

Such an amount can be administered each day, for example in individual doses administered once, twice, or three or more times a day. However, dosages stated herein on a per day basis should not be construed to require administration of the daily dose each and every day. For example, if the ambrisentan is provided in a suitably slow-release form, two or more daily dosage amounts can be administered at a lower frequency, e.g., as a depot every second day to once a month or even longer. Most typically and conveniently for the patient, ambrisentan is administered once a day, for example in the morning.

The ambrisentan can be administered for an extended treatment period. Typically, the longer the treatment continues, the greater and more lasting will be the benefits. Illustratively, the treatment period can be at least about one month, for example at least about 3 months, at least about 6 months or at least about 1 year. In some cases, administration can continue for substantially the remainder of the life of the subject.

In this and other embodiments, ambrisentan can be administered by any suitable route including oral, rectal, intranasal, intrapulmonary (e.g., by inhalation) or parenteral (e.g., intradermal, transdermal, subcutaneous, intramuscular or intravenous) routes. Oral administration is most convenient for the majority of subjects and can occur independently of meal times, i.e., with or without food.

In various embodiments, the subject experiences, during or following the treatment period, at least one of:
(a) adjustment of one or more hemodynamic parameters indicative of the pulmonary hypertension condition towards a more normal level versus baseline;
(b) increase in exercise capacity versus baseline;
(c) lowering of BDI versus baseline;
(d) improvement of one or more quality of life parameters versus baseline; and/or
(e) movement to a lower WHO functional class.

Any suitable measure of exercise capacity can be used; a particularly suitable measure is obtained in a 6-minute walk test (6MWT), which measures how far the subject can walk in 6 minutes, i.e., the 6-minute walk distance (6MWD).

The Borg dyspnea index (BDI) is a numerical scale for assessing perceived dyspnea (breathing discomfort). It measures the degree of breathlessness after completion of the 6MWT, where a BDI of 0 indicates no breathlessness and 10 indicates maximum breathlessness.

In various embodiments, ambrisentan can be administered in an amount effective to adjust one or more hemodynamic parameters indicative of the pulmonary hypertension condition towards a more normal level. In one such aspect, mean PAP is lowered, for example by at least about 3 mmHg, or at least about 5 mmHg, versus baseline. In another such aspect, PVR is lowered or PCWP or LVEDP is lowered. In yet another such aspect, cardiac output or cardiac index is raised.

In various embodiments, ambrisentan can be administered in an amount effective to improve cardiopulmonary function versus baseline. Any measure of cardiopulmonary function can be used; illustratively 6MWD is increased or BDI is lowered.

In one such aspect, 6MWD is increased from baseline by at least about 10 m, for example at least about 20 m or at least about 30 m. In many instances, the method of the present embodiment will be found effective to increase 6MWD by as much as 50 m or even more.

In another such aspect, BDI, illustratively as measured following a 6MWT, is lowered from baseline by at least about 0.5 index points. In many instances, the method of the present embodiment will be found effective to lower BDI by as much as 1 full index point or even more.

The SF-36® health survey provides a self-reporting, multi-item scale measuring eight health parameters: physical functioning, role limitations due to physical health problems, bodily pain, general health, vitality (energy and fatigue), social functioning, role limitations due to emotional problems, and mental health (psychological distress and psychological well-being). The survey also provides a physical component summary and a mental component summary (see, for example, Ware: SF-36® Health Survey Update, http://www.sf-36.org/tools/sf36.shtml). In various embodiments, ambrisentan can be administered in an amount effective to improve quality of life of the subject, illustratively as measured by one or more of the health parameters recorded in an SF-36® survey.

In another embodiment, a method is provided for improving the prognosis for a subject having a pulmonary hypertension condition. The method of this embodiment comprises administering to the subject ambrisentan at a dose and frequency and for a treatment period effective to provide (a) a reduction in probability of a clinical worsening event during the treatment period, and/or (b) a reduction from baseline in serum brain natriuretic peptide (BNP) concentration.

In a particular embodiment, the method is effective to provide a reduction of at least about 25%, for example at least about 50%, at least about 75% or at least about 80%, in probability of a clinical worsening event during the treatment period. Clinical worsening event (CWEs) include death, lung transplantation, hospitalization for the pulmonary hypertension condition, atrial septostomy, initiation of additional pulmonary hypertension therapy or an aggregate thereof. Therefore, the present embodiment provides a method effective to provide a reduction of at least about 25%, for example at least about 50%, at least about 75% or at least about 80%, in probability of death, lung transplantation, hospitalization for pulmonary arterial hypertension, atrial septostomy and/or initiation of additional pulmonary hypertension therapy during the treatment period. Time to clinical worsening of the pulmonary hypertension condition is defined as the time from initiation of an ambrisentan treatment regime to the first occurrence of a CWE. In an embodiment, the method improves exercise ability and delay clinical worsening without causing treatment-limiting liver injury.

In another particular embodiment, the method is effective to provide a reduction from baseline of at least about 15%, for example at least about 25%, at least about 50% or at least about 75%, in BNP concentration.

The pulmonary hypertension condition according to the second embodiment can comprise any one or more of the conditions in the WHO classification (e.g., Simonneau (2009) JACC 54:S43-54) described above. In one aspect of the second embodiment, the condition comprises PAH (WHO Group 1), for example idiopathic PAH, heritable PAH or PAH associated with another disease.

In another embodiment, a method is provided for prolonging the life of a subject having a pulmonary hypertension condition, comprising administering to the subject ambrisentan at a dose and frequency and for a treatment period effective to increase life expectancy, from a time of initiation of treatment, by at least about 30 days. Variants and illustrative modalities of this method are as set forth for the second embodiment above.

In any of the methods described hereinabove, the subject can be male or female. For example, ambrisentan can be administered to a female subject according to any of the above methods, including the indicated variants and illustrative modalities thereof. Alternatively, ambrisentan can be administered to a male subject, for example a reproductively active male subject, according to any of the above methods, including the indicated variants and illustrative modalities thereof.

In yet another embodiment, a method is provided for treating a pulmonary hypertension condition in a reproductively active male subject, the method comprising administering a therapeutically effective amount of ambrisentan to the subject, wherein fertility of the subject is not substantially compromised. “Not substantially compromised” in the present context means that spermatogenesis is not substantially reduced by the treatment and that no hormonal changes are induced that are indicative of or associated with reduced spermatogenesis. Male fertility can be assessed directly, for example, by sperm counts from semen samples, or indirectly by changes in hormones such as follicle stimulating hormone (FSH), luteinizing hormone (LH), inhibin B and testosterone.

In yet another embodiment, a method is provided for treating a pulmonary hypertension condition classified in WHO Groups 2-5 in a subject, comprising administering a therapeutically effective amount of ambrisentan to the subject.

In yet another embodiment, a method is provided for treating PAH in a subject, comprising administering a therapeutically effective amount of ambrisentan to the subject, wherein the PAH is associated with one or more of (a) a congenital heart defect, (b) portal hypertension, (c) use of a drug or toxin other than an anorexigen, (d) thyroid disorder, (e) glycogen storage disease, (f) Gaucher disease, (g) hereditary hemorrhagic telangiectasia, (h) hemoglobinopathy, (i) myeloproliferative disorder, (j) splenectomy, (k) pulmonary veno-occlusive disease and/or (l) pulmonary capillary hemangiomatosis. Variants and illustrative modalities of this method are as set forth hereinabove.

In one embodiment, the pulmonary hypertension condition comprises left-sided atrial or ventricular heart disease and/or left-sided valvular heart disease.

In another embodiment, the pulmonary hypertension condition is associated with one or more of chronic obstructive pulmonary disease (COPD), sleep-disordered breathing, an alveolar hypoventilation disorder, chronic exposure to high altitude, a developmental abnormality, thromboembolic obstruction of proximal and/or distal pulmonary arteries, a non-thrombotic pulmonary embolism, sarcoidosis, histiocytosis X, lymphangiomatosis, and/or compression of pulmonary vessels.

Pulmonary Hypertension Treatment without Companion Diagnosis

Endothelin-1 (ET-1) is a peptide made in the endothelium which can constrict blood vessels and elevate blood pressure. ET-1 concentrations are increased in plasma and lung tissue of patients with PAH. As a result, endothelin receptor antagonists (ERAs) have been developed to treat PAH. ERAs have been found that are therapeutically effective in treating patients with PAH. However, this class of drug compounds has been found to be capable of elevating liver aminotransferases and bilirubin, which are potential signs of hepatotoxicity. In other words, hepatotoxicity is a known risk of all ERAs.

The first ERA approved for treating PAH was bosentan (Tracleer®). This compound was approved by the FDA in 2001, with a boxed warning that elevations of liver aminotransferases, e.g., ALT (alanine aminotransferase) and AST (aspartate aminotransferase), and liver failures have been reported (see FDA approved Prescribing Label for bosentan (Tracleer®) revised February 2011). Because of the risks of liver injury and teratogenicity, bosentan is only available through a restricted distribution program, known as Tracleer Access Program (TAP). This program requires mandatory liver function tests (LFTs), evaluating ALT, AST and bilirubin, prior to prescribing the drug and at regular monthly intervals thereafter. This type of frequent testing becomes burdensome for many patients, especially if the patient is required to remain on the treatment for many years. Furthermore, the drug is not recommended for patients with moderate or severe liver impairment. It is reported that even with regular liver testing, liver cirrhosis and liver damage can still occur with bosentan treatment. Therefore, the inconvenience of frequent testing, coupled with a restricted class of suitable patients, leaves much to be desired for the treatment of PAH with bosentan.

Sitaxsentan (Thelin®) is another ERA that was in development for treating PAH. Similar to bosentan, sitaxsentan also was found to cause liver damages, elevating ALT and AST. Patients are not allowed to take sitaxsentan if AST and/or ALT levels are >3×ULN or if direct bilirubin>2×ULN. The drug sponsor (Pfizer, Inc.) has found that idiosyncratic hepatotoxicity caused by sitaxsentan is not associated with identifiable risk factors and is unlikely to be detected by routine monitoring. Furthermore, the drug sponsor discovered two fatalities associated with hepatic injury in patients undergoing treatment with sitaxsentan. As a result of the discovered hepatotoxicity, the drug sponsor has discontinued clinical trials in all countries including the U.S. and Japan Phase 3 registration trials.

Ambrisentan (Letairis®) is a propanoic acid-based ERA, selective for the endothelin type A (ETA) receptor. Ambrisentan was approved by the FDA in 2007 for the treatment of PAH. The U.S. prescribing information includes a boxed warning, describing the potential for liver injury.

Ambrisentan, because of the potential risk of hepatotoxicity, was approved with a Risk Evaluation and Mitigation Strategy (REMS) program. The REMS program (known as the Letairis Education and Access Program [LEAP]) involves the use of a targeted education and outreach program to prescribers and patients and a performance-linked, closed distribution system for dispensing drug through participating specialty pharmacies. A key element of the REMS includes mandatory monthly aminotransferase testing, with follow-up to ensure compliance before prescriptions are refilled.

Under LEAP requirements, prescribing physicians and patients must fill out and sign an enrolment and consent form, agreeing to: discuss the risks of ambrisentan with each patient, review the patient Medication Guide and patient education brochure with each patient, order and review liver function tests (ALT, AST and bilirubin), prior to initiating treatment and monthly during treatment, notify LEAP of any adverse events, and agree to re-enrol appropriate patients after the first 6 months and annually thereafter by completing and submitting new enrolment forms.

To help overcome some of the burden, on patients and prescribers, of scheduling and completing required companion diagnostic testing, the manufacturer of ambrisentan instituted a voluntary program called LabSync that coordinates the monthly blood draws, reminds patients of upcoming appointments, and provides prescribers with access to laboratory test results in a centralized database.

It has been consistently reported that ERAs are hepatotoxic in PAH patients, with no established mechanism for the hepatotoxicity except that these compounds are endothelin antagonists. Further, because of the risk for hepatotoxicity, FDA has only approved ERAs with an absolute requirement for patients and physicians to enrol in a Risk Evaluation and Mitigation program requiring monthly use of companion diagnostics to monitor liver function. In addition, patients displaying moderate or severe liver impairment may not be treated for PAH with an ERA. Prior to the Mar. 4, 2011 change to the black box warning in the Letairis label, which removed the requirement for monthly measurement of liver aminotransferase levels during ambrisentan treatment, there was no known ERA that could be legally prescribed without mandatory drug labeling instruction to measure liver aminotransferase levels during ERA treatment.

However, it has been surprisingly demonstrated that ambrisentan can be administered safely and effectively for treating pulmonary hypertension in a subject, without drug labeling instruction to monitor liver aminotransferase levels during ambrisentan treatment. As a result, ambrisentan patients and prescribing physicians no longer have to spend unnecessary time, money and the discomfort of going through FDA required monthly monitoring and evaluation of liver function tests, using companion diagnostics to measure, for example, alkaline phosphatase (ALK-P), ALT, AST and/or bilirubin levels. In addition, patients with mild hepatic impairment are no longer automatically disqualified from receiving ambrisentan, which can be a life-saving therapy in PAH patients. The absence of a requirement, in the currently approved label of ambrisentan, to monitor liver function is the result of unexpected findings from comparing ambrisentan to other ERAs and importantly, the result of post marketing studies which demonstrate to the satisfaction of the FDA that such monitoring is not warranted with respect to ambrisentan. Prior to these unexpected results and REMS findings, all previous FDA approved ERAs had such a requirement in the approved prescribing information label.

Therefore, in an embodiment, there is provided a method for treating pulmonary hypertension in a subject, comprising administering to the subject a daily dose of ambrisentan from 1 mg to about 15 mg, wherein the method is carried out without drug labeling instruction to monitor liver aminotransferase levels, except as clinically indicated, during ambrisentan treatment. In various embodiments, the daily dose of ambrisentan is from about 2.5 mg to less than about 5 mg, such as about 2.5 mg and 5 mg. In a particular embodiment, the daily dose of ambrisentan is about 5 mg, and the daily dose is increased from about 5 mg to about 10 mg if the about 5 mg ambrisentan daily dose is tolerated by the subject.

Ambrisentan treatment of this disclosure can be carried out without drug labeling instruction to monitor liver aminotransferase levels, but a medical professional such as a doctor may instruct a patient to take a liver toxicity monitoring program at his or her discretion, as clinically indicated.

In an embodiment, a subject having mild hepatic impairment is treated with the method of the present disclosure using ambrisentan. The subject having a mild liver abnormality may be diagnosed with a grade 1 abnormality in one or more biomarkers of liver function. Liver function impairment can be assessed by abnormal laboratory values associated with liver function as shown in Tables for Laboratory Abnormalities in “Guidance for Industry—Toxicity Grading Scale for Healthy Adult and Adolescent Volunteers Enrolled in Preventive Vaccine Clinical Trials” FDA, September 2007. Pertinent part of the table is reproduced below.

Laboratory abnormalities associated with liver function

| | | | | Potentially Life |
|---|---|---|---|---|
| | Mild | Moderate | Severe | Threatening |
| Serum | (Grade 1) | (Grade 2) | (Grade 3) | (Grade 4) |
| Liver Function Tests - | 1.1-2.5 × ULN* | 2.6-5.0 × ULN | 5.1-10 × ULN | >10 × ULN |
| ALT, AST increase by | | | | |
| factor | | | | |
| Bilirubin - when | 1.1-1.25 × ULN | 1.26-1.5 × ULN | 1.51-1.75 × ULN | >1.75 × ULN |
| accompanied by any | | | | |
| increase in Liver | | | | |
| Function Test increase | | | | |
| by factor | | | | |
| Alkaline phosphatase | 1.1-2.0 × ULN | 2.1-3.0 × ULN | 3.1-10 × ULN | >10 × ULN |
| increase by factor | | | | |

*ULN is the upper limit of the normal range

In another embodiment, there is provided a method for treating pulmonary hypertension in a subject, comprising administering to the subject a daily dose of ambrisentan from 1 mg to about 15 mg, wherein the subject in need thereof has exhibited a grade 2, 3 or 4 abnormality in one or more biomarkers of liver function, and wherein the method is carried out without drug labeling instruction to monitor liver aminotransferase levels during ambrisentan treatment. In an embodiment, the ambrisentan administration is discontinued until a biomarker of liver function is within a normal limit.

In a particular embodiment, ambrisentan is administered to a subject who has exhibited a grade 2 abnormality in one or more biomarkers of liver function. In some embodiment, the subject may be a patient who has exhibited a grade 2 abnormality in one or more biomarkers of liver function after previous ERA therapy, such as bosentan or sitaxsentan treatment.

### Combination Therapy

In all the above embodiments, the ambrisentan can be administered in monotherapy.

Alternatively, the ambrisentan can be administered in combination therapy with a second active agent effective for the treatment of the pulmonary hypertension condition or a condition related thereto.

The term “combination therapy” (or “co-therapy”), in defining use of ambrisentan and a second active agent, as described herein, is intended to embrace administration of each agent in a sequential manner in a regimen that will provide beneficial effects of the drug combination, and is intended as well to embrace co-administration of these agents in a substantially simultaneous manner, such as by oral ingestion of a single capsule having a fixed ratio of these active agents or ingestion of multiple, separate capsules for each agent. “Combination therapy” will also include simultaneous or sequential administration by intravenous, intramuscular or other parenteral routes into the body. Sequential administration also includes drug combination where the individual elements may be administered at different times and/or by different routes but which act in combination to provide a beneficial effect. It is expected that this combination therapy of ambrisentan and a second active agent will result in co-action of the ambrisentan and the second active agent, providing a pharmacokinetic interaction, or a pharmacodynamic interaction, or both, where the compounds are administered either simultaneously or sequentially, to permit such co-action.

When ambrisentan is administered concomitantly with a second active agent, one of skill in the art can readily identify a suitable dose for any particular second active agent from publicly available information in printed or electronic form, for example on the internet. Illustratively and without limitation, the ambrisentan can be administered with a second active agent comprising at least one drug selected from the group consisting of prostanoids, phosphodiesterase inhibitors (especially, phosphodiesterase-5 (PDE5) inhibitors), tyrosine kinase inhibitors, guanylate cyclase activators (such as, for example giociguat), calcium channel blockers, diuretics, anticoagulants, oxygen, NO (nitric oxide)-releasing compounds and combinations thereof.

Examples of drugs useful in combination therapy with ambrisentan are classified and presented in several lists below. Some drugs are active at more than one target; accordingly certain drugs may appear in more than one list. Use of any listed drug in a combination is contemplated herein, independently of its mode of action.

A suitable prostanoid can be illustratively selected from the following list: cicaprost, selexipag, alprostadil and prostacyclin. In particular embodiments, the prostanoid is selected from prostacyclin which includes, for example, beraprost, iloprost, epoprostenol and treprostinil.

A suitable PDE5 inhibitor can illustratively be selected from the following list: tadalafil, avanafil, lodenafil, mirodenafil, sildenafil, vardenafil and udenafil and salts thereof.

A suitable tyrosine kinase inhibitor can be illustratively selected from the following list: Imatinib, sorafenib, sunitanib, nilotinib, semaxinib, lestaurtinib, tandutinib, gefitinib and midostaurin.

A suitable calcium channel blocker can illustratively be selected from the following list:

### Arylalkylamines:

bepridil, clentiazem, diltiazem, fendiline, gallopamil, mibefradil, prenylamine, semotiadil, terodiline and verapamil;

### Dihydropyridine Derivatives:

amlodipine, aranidipine, azelnidipine, barnidipine, benidipine, cilnidipine, clevidipine, efonidipine, elgodipine, felodipine, isradipine, lacidipine, lercanidipine, manidipine, nicardipine, nifedipine, nilvadipine, nimodipine, nisoldipine, nitrendipine and pranidipine;

### Piperazine Derivatives:

cinnarizine, dotarizine, flunarizine, lidoflazine, lomerizine and manidipine; and

### Unclassified:

bencyclane, etafenone, fantofarone, monatepil and perhexyline.

Particularly suitable calcium channel blockers include amlodipine, diltiazem, felodipine, isradipine, nicardipine, nifedipine, nisoldipine, verapamil and combinations thereof.

A suitable diuretic can illustratively be selected from the following list:

### Organomercurials:

Chlormerodrin, chlorothiazide, chlorthalidone, meralluride, mercaptomerin sodium, mercumatilin sodium, mercurous chloride and mersalyl;

### Purines:

adenine, caffeine, guanine, hypoxanthine, isoguanine, pamabrom, protheobromine, theobromine, uric acid and xanthine;

### Steroids:

canrenone, oleandrin and spironolactone;

### Sulfonamide Derivatives:

acetazolamide, ambuside, butazolamide, chloraminophenamide, clofenamide, clopamide, clorexolone, disulfamide, ethoxzolamide, mefruside, methazolamide, tripamide and xipamide;

### Loop Diuretics:

azosemide, bumetanide, ethacrynic acid, etozolin, furosemide, piretanide, and torsemide;

### Thiazides and Analogs Thereof:

althiazide, bendroflumethiazide, benzthiazide, benzylhydrochlorothiazide, buthiazide, chlorthalidone, cyclopenthiazide, cyclothiazide, ethiazide, fenquizone, hydrochlorothiazide, hydroflumethiazide, indapamide, methyclothiazide, metolazone, paraflutizide, polythiazide, quinethazone, teclothiazide and trichlormethiazide;

### Uracil:

aminometradine;

### Osmotic Diuretics:

mannitol;

### Potassium-Sparing Diuretics:

amiloride and triamterene; and

### Unclassified:

Naxifylline, chlorazanil, ethacrynic acid, etozolin, isosorbide, Kiowa Hakko KW 3902, muzolimine, perhexyline, satavaptan, ticrynafen and urea.

In some embodiments, the diuretic if present comprises a thiazide or loop diuretic. Thiazide diuretics are generally not preferred where the patient has a complicating condition such as diabetes or chronic kidney disease, and in such situations a loop diuretic can be a better choice.

Particularly suitable thiazide diuretics include chlorothiazide, chlorthalidone, hydrochlorothiazide, indapamide, metolazone, polythiazide and combinations thereof. Particularly suitable loop diuretics include bumetanide, furosemide, torsemide and combinations thereof.

A suitable anticoagulant can illustratively be selected from the following list: acenocoumarol, ancrod, anisindione, atromentin, bivalirudin, bromindione, clorindione, coumetarol, cyclocumarol, dextran sulfate sodium, dicumarol, diphenadione, ethyl biscoumacetate, ethylidene dicoumarol, fluindione, hementin, heparin, hirudin, lepirudin, lumbrokinase, lyapolate sodium, nattokinase, pentosan polysulfate, phenindione, phenprocoumon, phosvitin, picotamide, tioclomarol, and warfarin.

In a particular embodiment, the combination therapy of this present disclosure comprises a second active agent which is selected from the group consisting of prostacyclin, PDE5 inhibitors, and guanylate cyclase activators.

Where the pulmonary hypertension condition is associated with an underlying disease (for example CTD, HIV infection or COPD), ambrisentan can optionally be administered in combination therapy with one or more drugs targeting the underlying condition.

When ambrisentan is used in combination therapy with one or more drugs, the ambrisentan and at least one drug can be administered at different times or at about the same time (at exactly the same time or directly one after the other in any order). The ambrisentan and the second active drug can be formulated in one dosage form as a fixed-dose combination for administration at the same time, or in two or more separate dosage forms for administration at the same or different times.

Separate dosage forms can optionally be co-packaged, for example in a single container or in a plurality of containers within a single outer package, or co-presented in separate packaging (“common presentation”). As an example of co-packaging or common presentation, a kit is contemplated comprising, in separate containers, ambrisentan and at least one drug useful in combination with the ambrisentan. In another example, the ambrisentan and the at least one drug useful in combination therapy with the ambrisentan are separately packaged and available for sale independently of one another, but are co-marketed or co-promoted for use according to the invention. The separate dosage forms can also be presented to a patient separately and independently, for use according to the invention.

Typically at least the ambrisentan is provided in an orally deliverable formulation, for example a formulation adapted for oral delivery of a ambrisentan dose of about 1 to about 15 mg/day, e.g., about 2.5 to about 5 mg/day. The ambrisentan formulation can be adapted for any suitable frequency of administration, but in one embodiment is adapted for once a day oral administration.

In one embodiment, at least one of the drugs other than ambrisentan in the combination is provided in an orally deliverable formulation; for example, each of the drugs can be so provided, and each of the drugs can be in a formulation adapted for once a day oral administration. Each of the drugs other than ambrisentan is typically present in the combination in an amount providing an adequate to full dose of the drug. One of skill in the art can readily identify a suitable dose for any particular drug from publicly available information in printed or electronic form, for example on the internet.

Any two or more drugs in the combination can optionally be co-formulated to provide a fixed dose combination. For example, the ambrisentan can be co-formulated with any one or more of the other drugs in the combination.

Mention of a particular drug or second active agent in the present specification and claims will be understood, except where the context demands otherwise, to include pharmaceutically acceptable salts, esters, prodrugs, metabolites, racemates and enantiomers of the drug, to the extent that such salts, esters, prodrugs, metabolites, racemates or enantiomers exist and are therapeutically effective.

In an embodiment, there is provided a method of treating or preventing pulmonary hypertension comprising administration of therapeutic amounts of ambrisentan and a PDE5 inhibitor, wherein the method is carried out without drug labeling instruction to monitor liver aminotransferase levels, except as clinically indicated, during ambrisentan treatment. In a particular aspect, the method comprises administration of a therapeutic amount of tadalafil or a salt thereof and a therapeutic amount of ambrisentan or a salt thereof. The two agents may be administered separately or together in separate or a combined dosage unit. If administered separately, the ambrisentan may be administered before or after administration of the tadalafil.

It is discovered that there is co-action of the combination of ambrisentan and tadalafil to relax endothelin-induced contractions and to inhibit hypoxia-induced pulmonary arterial pressure (PAP) in a PAH animal model. Such enhanced efficacy of the co-action is apparent as the combined effect is greater than the additive effects of mono-administration of each drug. In one aspect, such enhanced efficacy amounts to at least about 5% enhanced effectiveness over the additive effectiveness of mono-administration of each drug. Alternatively, such enhancement is at least about 10%, 15%, 20%, 25%, 30%, 35%, 40%, 45%, 50%, 55%, 60%, 65%, 70%, 80%, 90% or 100%. In other words, the combinations can achieve an effectiveness that is at least about 5%, or alternatively 10%, 15%, 20%, 25%, 30%, 35%, 40%, 45%, 50%, 55%, 60%, 65%, 70%, 80%, 90% or 100% greater than the sum of effectiveness of mono-administrations of either agent.

The enhanced effect of the co-action of ambrisentan and a PDE5 inhibitor may depend on the amounts of each individual agent and/or ratios of such amounts. In one aspect, the ratio of the amount of ambrisentan and the amount of the PDE5 inhibitor, in order to achieve such enhanced effects, can be from about 2:1, or alternatively 1:1, 1:1.5, 1:2, 1:2.5, 1:3, 1:4, 1:5, 1:6, 1:7, 1:8, 1:9 or 1:10 to about 1:3, or alternatively about 1:4, 1:5, 1:6, 1:7, 1:8, 1:9, 1:10, 1:11, 1:12, 1:15 or 1:20. In one aspect, the ratio of amounts is a ratio of molar amounts of each agent. In another aspect, the ratio of amounts is a weight ratio of each agent.

In some embodiments, the ratio of the amount of ambrisentan and the amount of the PDE5 inhibitor, in order to achieve enhanced effects, is around 1:3, which, for instance, can be from about 1:1.5 to about 1:5, or alternatively from about 1:2 to about 1:4. In one aspect, the ratio is a weight ratio of each agent. In one aspect, the amount of ambrisentan or a salt thereof is from about 5 mg to about 10 mg daily for a human subject. In another aspect, the amount of tadalafil or a salt thereof is from about 15 mg to about 30 mg daily for a human subject.

In some embodiments, the ratio of the amount of ambrisentan and the amount of tadalafil, in order to achieve enhanced effects, is around 1:1, which, for instance, can be from about 2:1 to about 1:2, or alternatively from about 1:1 to about 1:2. In another aspect, the ratio is a weight ratio of each agent. In one aspect, the amount of ambrisentan or a salt thereof is from about 5 mg to about 10 mg daily for a human subject. In another aspect, the amount of tadalafil or a salt thereof is from about 5 mg to about 10 mg daily for a human subject.

In some embodiments, the ratio of the amount of ambrisentan and the amount of tadalafil, in order to achieve enhanced effects, is around 1:10, which, for instance, can be from about 1:5 to about 1:15, or alternatively from about 1:8 to about 1:12. In another aspect, the ratio is a weight ratio of each agent. In one aspect, the amount of ambrisentan or a salt thereof is from about 2 mg to about 5 mg daily for a human subject. In another aspect, the amount of tadalafil or a salt thereof is from about 20 mg to about 40 mg daily for a human subject.

In some embodiments, the ratio of the amount of ambrisentan and the amount of tadalafil, in order to achieve enhanced effects, is around 1:4, which, for instance, can be from about 1:2 to about 1:7, or alternatively from about 1:3 to about 1:5. In another aspect, the ratio is a weight ratio of each agent. In one aspect, the amount of ambrisentan or a salt thereof is from about 5 mg to about 10 mg daily for a human subject. In another aspect, the amount of tadalafil or a salt thereof is from about 30 mg to about 40 mg daily for a human subject.

In some embodiments, the ratio of the amount of ambrisentan and the amount of tadalafil, in order to achieve enhanced effects, is around 1:8, which, for instance, can be from about 1:5 to about 1:10, or alternatively from about 1:7 to about 1:9. In another aspect, the ratio is a weight ratio of each agent. In one aspect, the amount of ambrisentan or a salt thereof is from about 2 mg to about 5 mg daily for a human subject. In another aspect, the amount of tadalafil or a salt thereof is from about 30 mg to about 40 mg daily for a human subject.

### EXAMPLES

The following examples are merely illustrative, and do not limit this disclosure in any way.

The ambrisentan clinical development program included placebo-controlled and non-placebo-controlled studies. One Phase 2 study (AMB-220) and two Phase 3 studies (AMB-320 and AMB-321) were completed at the time of the NDA submission. Three ongoing studies (AMB-220-E, AMB-222, and AMB-320/321-E) provided additional data in both the original NDA and the 4-month safety update, and data from one additional ongoing study (AMB-323) were included in the 4-month safety update.

An assessment of hepatic events (including adverse events and elevated aminotransferase levels) from the post-marketing ambrisentan data showed an incidence of approximately 2%, which is consistent with the background rate of hepatic events observed in patients with PAH (not treated with ERAs). These events are typically related to the patients' underlying PAH or other comorbidity.

### Example 1

### Hepatic Safety Profile of Ambrisentan in Patients with PAH

### Cumulative Incidence of Aminotransferase Elevation

The cumulative incidence of aminotransferase elevations by severity, summarized as a percentage of the cumulative number of subjects who received ambrisentan in clinical trials, is provided in Table 1. The cumulative incidence of aminotransferase elevations>3×ULN (upper limit of the normal range) accompanied by total bilirubin>2×ULN was 0.3% (2 subjects, both with alternative causes for the liver function test (LFT) abnormalities); the associated mean exposure to ambrisentan was 112.5 weeks (maximum, 341.0 weeks). In comparison, during the 12-week placebo controlled trials, 1 patient (1/132, 0.7%) receiving placebo had elevated aminotransferases (both alanine transaminase (ALT) and aspartate transaminase (AST) were >5×ULN) along with elevated total bilirubin (>2×ULN).

TABLE 1
Distribution of Treatment-emergent Serum Aminotransferase
Abnormalities >3 × ULN in Ambrisentan-Treated Subjects

| | All Subjects (N = 707) |
|---|---|
| ALT/AST Elevations | Patient number (%) |
| Total >3 × ULN | 35 (5.0) |
| >3 to ≦5 × ULN | 25 (3.5) |
| >5 to ≦8 × ULN | 1 (0.1) |
| >8 × ULN | 9 (1.3) |
| >3 × ULN and total bilirubin >2 × ULN | 2 (0.3) |

Aminotransferase Elevations with Associated Elevation of Bilirubin:

As noted in Table 1, during clinical trials and long-term follow-up, two ambrisentan-treated subjects had aminotransferase elevations>3×ULN with concurrent elevations of total bilirubin>2×ULN. In comparison, one placebo-treated subject from these studies had similar concurrent elevations.

The first ambrisentan case was confounded by documented concurrent marked right heart failure and diagnosed after the patient admitted missing several doses of ambrisentan and 3-4 days of sildenafil and furosemide. The patient's baseline serum total bilirubin was 1.7×ULN before starting ambrisentan treatment.

The second ambrisentan case was confounded by intercurrent pneumonia, and ambrisentan was successfully re-started in this subject following resolution of the pneumonia and the LFT abnormalities. The subject's LFTs were not reported to be elevated for the ensuing 177 days of ambrisentan treatment.

### Example 2

### Hepatic Safety Profile of Ambrisentan in Patients with PAH

Patients with Prior Aminotransferase Elevations while Receiving Sulfonamide-Based ERA Therapy Who were then Treated with Ambrisentan

Studies were conducted to investigate the effects of ambrisentan in subjects who had discontinued bosentan, sitaxsentan or both due to aminotransferase elevations>3×ULN. They were enrolled in studies AMB-222 (n=36) and AMB-323 (n=27). Of these subjects, 97% (35 of 36 subjects) in AMB-222 and 89% (24 of 27 subjects) in AMB-323 did not experience aminotransferase elevations while receiving ambrisentan in these studies. The remaining subjects in these studies (a total of 4 subjects) did have an aminotransferase level>3×ULN and are included in Table 1.

In two of the four subjects, the aminotransferase elevations normalized with no change in ambrisentan therapy. In one of the four subjects, the aminotransferase elevations normalized on a reduced dose of ambrisentan therapy and remained within normal range after the ambrisentan dose was later increased. In the last of the four subjects, the LFTs remained elevated but at a level less than 3×ULN (ALT 2.4×ULN, AST 1.7×ULN, and alkaline phosphatase (ALK-P) 1.7×ULN) approximately 9 weeks after discontinuing ambrisentan therapy. Of note, this last subject's aminotransferase elevations occurred approximately 15.5 months after starting ambrisentan therapy. In summary, none of the 4 cases showed clear evidence of a role for ambrisentan in the elevated serum aminotransferase levels.

### Example 3

### Hepatic Safety Profile of Ambrisentan in Patients with PAH

eDISH Plots of Clinical Trial Data

eDISH (evaluation of drug-induced serious hepatotoxicity) plots compare maximum bilirubin values to maximum ALT or AST values. Plots are divided into quadrants by superimposing lines corresponding to 2×ULN for bilirubin and 3×ULN for ALT or AST. The two right quadrants identify subjects with potential liver injury; the lower right quadrant includes subjects with ALT or AST>3×ULN. In these plots, patients are only included once and the highest bilirubin is plotted against the highest ALT/AST, without necessarily having a temporal relationship.

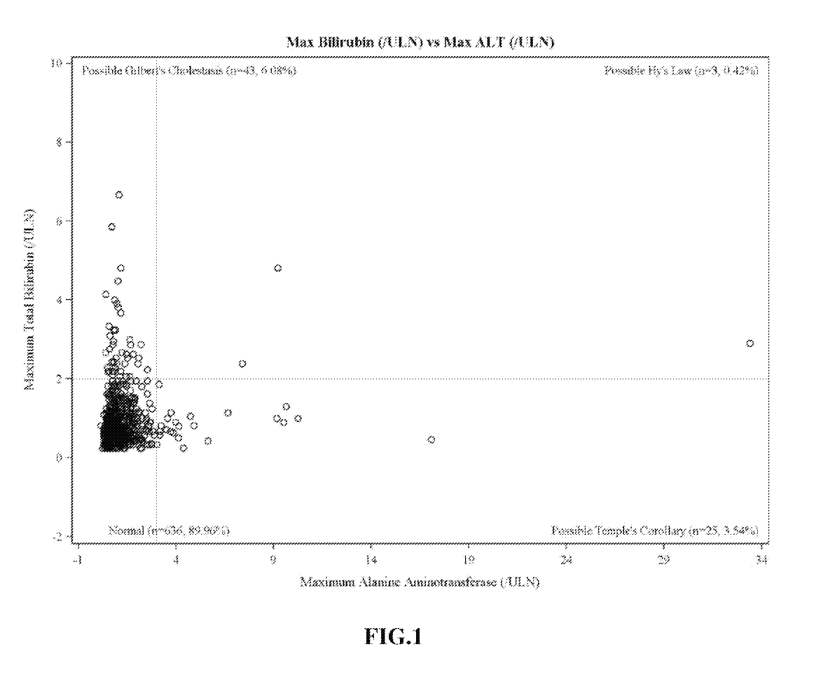

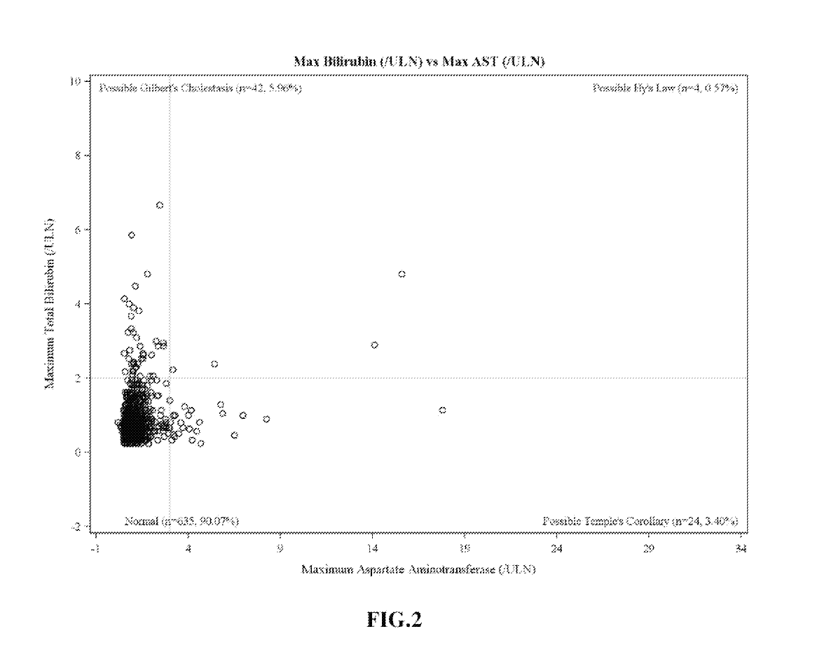

eDISH plots were prepared from clinical trial data. FIG. 1 shows eDISH plot comparing maximum bilirubin values to maximum ALT. FIG. 2 shows eDISH plot comparing maximum bilirubin values to maximum AST. The three subjects (2 ambrisentan-treated and 1 placebo-treated) with concurrent bilirubin (>2×ULN) and aminotransferase elevations (>3×ULN) noted in EXAMPLE 1, hereinabove, appear in the upper right hand quadrant of both plots. As is evident from FIG. 1 and FIG. 2, the vast majority of patients had bilirubin and aminotransferase values that remained in the normal range during the clinical studies and long-term follow-up.

### Example 4

### Spontaneous and Clinical Serious Adverse Event Data Received During the 2-Year Period after First Approval (15 Jun. 2007-14 Jun. 2009)

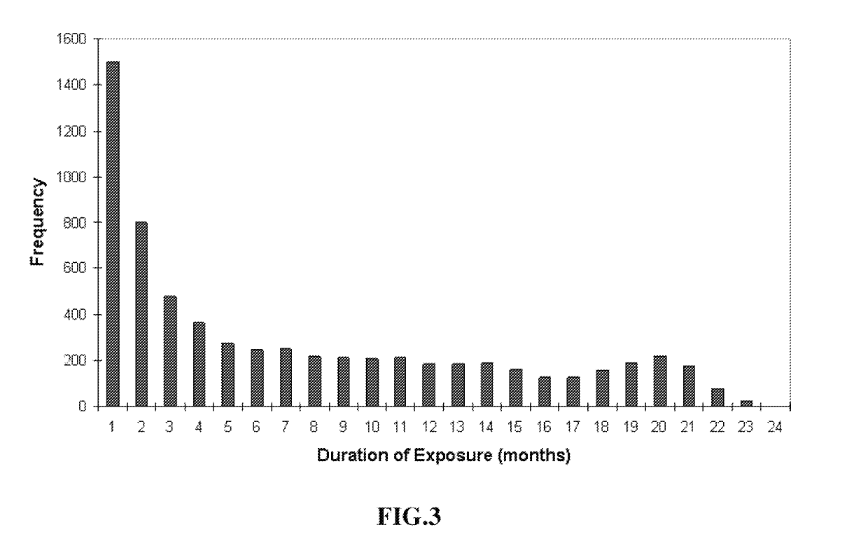

During the 2-year period since ambrisentan was first approved, the vast majority of patient exposure had been in the U.S. through Letairis Education and Access Program (LEAP). As of 14 Jun. 2009, a total of 6,622 patients had enrolled in LEAP and received at least one shipment of ambrisentan. Approximately half of the patients for whom duration of therapy was available received ambrisentan for a period of at least 6 months, resulting in a total of 3,847 patient-years of exposure. Exposure duration is depicted in FIG. 3 showing LEAP patients by exposure duration.

A total of 120 post-marketing spontaneous reports were identified for the 2-year period (15 Jun. 2007 through 14 Jun. 2009) where 119 reports were received via LEAP in the U.S. and the remaining one was received from Germany (outside of LEAP). As of 31 Mar. 2009, there were 118 patient-years of exposure to ambrisentan. All reports were actively followed up in accordance with Gilead Drug Safety procedures to obtain all important information needed to assess a potential relationship with ambrisentan therapy, particularly laboratory data, medical history and concurrent medications. All reports included potential alternative causes for the hepatic events which are unrelated to ambrisentan therapy, such as right-sided heart failure due to underlying PAH, other co-morbidities or concurrent medications.

Of the 120 spontaneous reports, 55 reports were medically-confirmed suspected adverse reaction reports. A total of 28 cases were considered “clinically significant hepatic events” which are defined as (1) ALT and/or AST increases >3×ULN, (2) serum total bilirubin increases >2×ULN, (3) any hepatic event associated with potential signs/symptoms of hepatic disease, or (4) any clinically significant hepatic event term (e.g., liver failure). These cases included 24 reports of serum aminotransferase (ALT/AST) increases >3×ULN; 3 reports of elevated serum total bilirubin>2×ULN; and 1 report of elevated serum aminotransferases with signs and symptoms, but AST and ALT values were not provided. Of these 28 cases, there were 3 reports of both elevated ALT/AST>3×ULN and serum total bilirubin>2×ULN. However, these reports included probable alternate causes of the hepatic events such as sickle cell disease in a patient with baseline serum total bilirubin of ‘7.7’, suspected gall bladder disease in a patient who developed increased serum total bilirubin (5×ULN) and alkaline phosphatase (3.3×ULN) 11 days before an increase in ALT to 3.1×ULN, and documented cardiogenic shock and right-sided heart failure due to PAH.

Of the 120 spontaneous reports, there were 33 reports medically-confirmed, in which the healthcare provider specifically indicated that no causal association with ambrisentan was suspected. These 33 cases included 14 reports that were considered clinically significant, including two cases where ALT/AST was >3×ULN and serum total bilirubin>2×ULN. However, probable alternate causes of the hepatic events in these reports included underlying hepatitis C, stage 4 alcoholic cirrhosis, underlying portopulmonary hypertension, abrupt withdrawal of treprostinil in a patient with cirrhosis of the liver secondary to chronic hepatitis C infection and hepatocellular carcinoma status post chemoembolization.

Of the 120 spontaneous reports, 27 reports came from consumers via specialty pharmacies and were not medically-confirmed. None of these cases had any clear evidence of a suspected adverse reaction, and there were no cases with laboratory data indicating an ALT/AST of >3×ULN and serum total bilirubin>2×ULN. One of the 27 reports was considered clinically significant, which was portal hypertension confounded by a history of primary biliary cirrhosis.

Of the 120 spontaneous reports, 5 reports were not medically-confirmed suspected adverse reactions. These cases involved non-serious elevations in transaminase levels, with limited data upon which to assess drug causality. None of the 5 cases was considered clinically significant.

There were 7 drug-related serious adverse event (SAE) reports from clinical studies which had evidence of alternative causes of the hepatic events or were poorly documented. Six of the reports were considered clinically significant, including one case where ALT/AST was >3×ULN and serum total bilirubin>2×ULN. However, probable causes of the hepatic events in this case included concurrent right heart failure, diagnosed after the patient admitted missing several doses of ambrisentan and 3-4 days of sildenafil and furosemide and with a baseline serum total bilirubin of 1.7×ULN.

The estimated post-marketing incidence rate of hepatic events with ambrisentan in the U.S. during the 2-year post-marketing period is 1.8% (119/6622), detected through LEAP. This rate is consistent with the background rate of placebo-treated PAH patients in clinical trials evaluating ERAs which appeared to have an incidence rate of 2%-3% over a 12- to 24-week period.

The post-marketing hepatic event data collected during the first 2 years of marketing is consistent with previous findings from clinical studies. The data do not support an increased risk of hepatic events with ambrisentan compared to the background rate of hepatic events in PAH patients.

### Example 5

### Hepatic Safety Profile of Ambrisentan in Patients with PAH

### Spontaneous Disproportionality Analysis Comparing Ambrisentan and Bosentan

A disproportionality analysis was performed for ambrisentan and bosentan using information from the FDA Adverse Events Reporting System (AERS) spontaneous reporting database. In the present analysis, the Bayesian approach of ‘Multi Item Gamma Poisson Shrinker’ (MGPS) was selected (DuMouchel, W., “Bayesian data mining in large frequency tables, with an application to the FDA spontaneous reporting system.” The American Statistician 53, 177-190 (1999); and Szarfman, et al., “Use of screening algorithms and computer systems to efficiently signal higher-than-expected combinations of drugs and events in the US FDA's spontaneous reports database.” Drug Safety 25, 381-392 (2002)), which derives the empiric Bayesian geometric mean (EBGM) and the corresponding 90% confidence interval (EB05, EB95). The value of the EBGM reflects the ratio of the number of observed reports of a drug-adverse event combination with the number expected under an assumption of independence between the drugs and events in the database. In addition, MGPS reduces the values of the Bayesian observed-to-expected ratios toward the null hypothesis value of 1 by an amount that depends on their statistical variability.

In order to reduce the occurrence of false positives due to age group or gender influence, the safety reports were stratified by subject age group and gender. For each drug, the AERS data were analyzed for the 18-month period following the respective approval dates for ambrisentan and bosentan: 15 Jun. 2007 to 14 Dec. 2008 for ambrisentan and 21 Nov. 2001 to 20 May 2003 for bosentan.

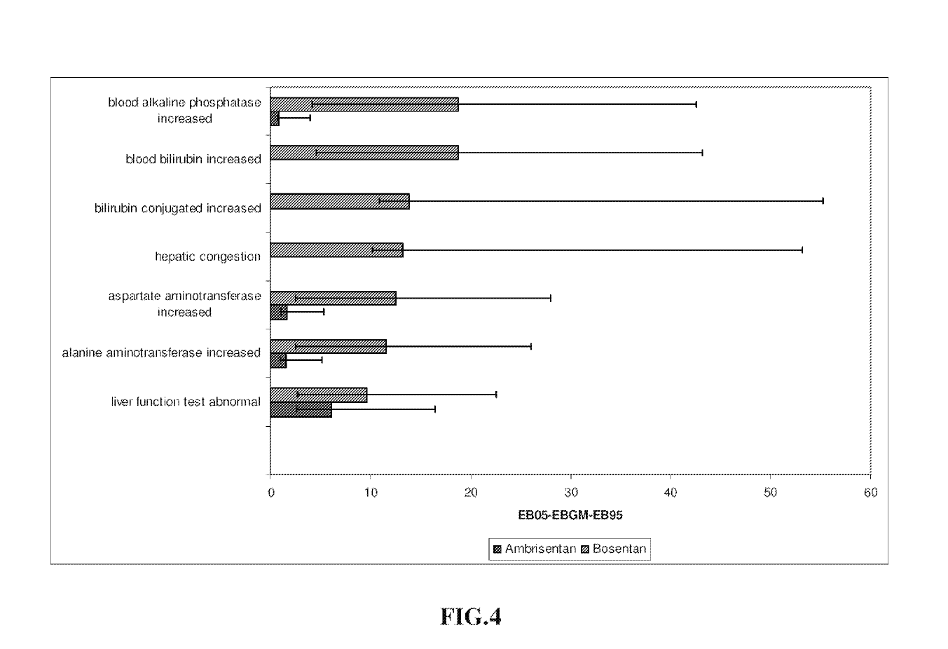

Data for hepatic adverse events are presented in Table 2 and FIG. 4. Only events with an EB05 score of >2 for either ambrisentan or bosentan were included. This threshold is generally accepted as a signal of disproportional reporting (Szarfman, et al., “Use of screening algorithms and computer systems to efficiently signal higher-than-expected combinations of drugs and events in the US FDA's spontaneous reports database.” Drug Safety 25, 381-392 (2002)). The comparator dataset in the calculations for ambrisentan and bosentan included data for all other drugs in the AERS database.

One hepatic event term, “liver function test abnormal,” for ambrisentan exceeded the EB05 threshold for a potential signal of disproportional reporting (with an EB05 of 3.5). In contrast, there were 7 hepatic event terms for bosentan with EB05 scores ranging between 6.9 (for ‘liver function test abnormal’) and 14.6 (for ‘blood alkaline phosphate increased’), indicating these events were reported at least 6.9 times more frequently for bosentan than would be expected based on the reporting of these events for other drugs in the AERS data set. These scores do not indicate a causal relationship between the drug and event, only that the event was reported more frequently than expected compared to other drugs in the database.

The disproportionality analysis of spontaneous FDA AERS data shows a stronger signal of disproportionate reporting of hepatic events for bosentan than for ambrisentan. This difference in spontaneous reporting is unlikely to be due to differences in the Risk Evaluation and Mitigation Strategy (REMS) programs for the two drugs since both programs involve regular contact with patients and the REMS providers due to the required monthly testing of liver function. No other therapeutic agents approved for treating PAH (e.g., sildenafil) were included in the analysis since none has REMS programs similar to ambrisentan and bosentan.

TABLE 2
MGPS Analysis of FDA AERS Data for Ambrisentan and Bosentan
Hepatic Events (18 Months Post Approval)

| | Ambrisentan | | | Bosentan | | |
|---|---|---|---|---|---|---|
| Hepatic Event | Count | EBGM | EB05-EB95 | Count | EBGM | EB05-EB95 |
| Blood alkaline | 1 | 0.86 | 0.16-3.15 | 46 | 18.79 | 14.63-23.82 |
| phosphate increased | | | | | | |
| Blood bilirubin | 0 | — | — | 37 | 18.75 | 14.16-24.43 |
| increased | | | | | | |
| Bilirubin conjugated | 0 | — | — | 5 | 13.91 | 3.04-41.30 |
| increased | | | | | | |
| Hepatic congestion | 0 | — | — | 5 | 13.18 | 2.96-39.98 |
| AST increased | 3 | 1.63 | 0.59-3.76 | 60 | 12.52 | 9.95-15.52 |
| ALT increased | 3 | 1.55 | 0.56-3.58 | 57 | 11.57 | 9.06-14.49 |
| Liver function test | 10 | 6.11 | 3.50-10.34 | 38 | 9.59 | 6.90-12.98 |
| abnormal | | | | | | |
| Ascites | 2 | 1.80 | 0.52-4.86 | 8 | 3.60 | 1.97-6.25 |
| Transaminases | 2 | 1.96 | 0.57-5.29 | 5 | 3.58 | 1.63-7.61 |
| increased | | | | | | |
| Blood bilirubin | 0 | — | — | 2 | 3.28 | 0.79-34.10 |
| unconjugated increased | | | | | | |
| INR increased | 1 | 0.78 | 0.14-2.82 | 9 | 3.08 | 1.75-5.13 |
| Hepatosplenomegaly | 0 | — | — | 2 | 2.09 | 0.66-5.55 |
| Hepatitis | 1 | 0.89 | 0.16-3.21 | 5 | 1.63 | 0.77-3.12 |
| Spider naevus | 0 | — | — | 1 | 1.46 | 0.32-4.92 |
| Hepatic infarction | 0 | — | — | 1 | 1.44 | 0.32-4.81 |
| Prothrombin time | 1 | 1.29 | 0.23-4.70 | 2 | 1.22 | 0.39-3.07 |
| prolonged | | | | | | |
| Hepatic failure | 0 | — | — | 3 | 1.07 | 0.41-2.37 |
| Hepatitis acute | 0 | — | — | 1 | 1.04 | 0.24-3.29 |
| Hepatic encephalopathy | 0 | — | — | 1 | 1.00 | 0.23-3.16 |
| Hepatic necrosis | 0 | — | — | 1 | 0.94 | 0.21-2.96 |
| Hepatic function | 0 | — | — | 2 | 0.91 | 0.29-2.30 |
| abnormal | | | | | | |
| Hepatomegaly | 0 | — | — | 1 | 0.79 | 0.18-2.50 |
| Liver disorder | 2 | 1.43 | 0.42-3.86 | 1 | 0.56 | 0.13-1.78 |
| Hepatic enzyme | 3 | 1.67 | 0.61-3.85 | 0 | — | — |
| increased | | | | | | |
| Hepatic steatosis | 1 | 1.06 | 0.19-3.85 | 0 | — | — |

Data sorted by EBGM (descending order) for Bosentan
EBGM = empirical Bayesian geometric mean and the respective upper and lower bounds of the 90% confidence interval (EB05 and EB95)

Based on the data obtained to date, ambrisentan has shown consistent safety results. Rates of hepatic events in ambrisentan-treated patients are consistent with background rates within the PAH population and are generally attributed to underlying disease, co-morbidities and/or concomitant mediations.

### Example 6

### Effect of Ambrisentan, Bosentan and Macitentan on Human Hepatic Uptake and Efflux Transporters

Fattinger, et al., Clin. Pharmacol. Ther. 69(4), 223-231 (2001) reports that a putative mechanism for hepatic adverse reactions observed with bosentan, a dual endothelin receptor antagonist, is inhibition of the hepatic transport of bile salts. Thus, three ERAs, bosentan, ambrisentan and macitentan, were tested for inhibition of hepatic transporters in vitro.

Macitentan is an experimental ERA in clinical development. Macitentan (Actelion-1 or ACT-064992), like bosentan, is a sulfonamide (IUPAC: [N-[5-(4-bromophenyl)-6-(2-(5-bromopyrimidin-2-yloxy)ethoxy)-pyrimidin-4-yl]-N′-propylaminosulfonamide]) and a dual endothelin receptor antagonist. Macitentan is also more lipophilic (octanol/aqueous buffer distribution coefficient, D=800:1) than bosentan (D=20:1) and ambrisentan (D=1:2.5) (Iglarz, et al., J. Pharmacol Exp. Ther. 327, 736-745 (2008)).

Inhibition constants (IC₅₀) were measured for human bile salt export pump (BSEP), sodium taurocholate cotransporting polypeptide (NTCP), multidrug resistance protein 2 (MRP2), P-glycoprotein (Pgp), breast cancer resistance protein (BCRP), organic anion-transporting polypeptide 1B1 (OATP1B1), and OATP1B3 in transfected cell-lines. Known inhibitors were used as positive controls. The transfected cell-lines and inhibitors used in this study are summarized in Table 3.

TABLE 3
Cell-lines and Inhibitors Used for Measuring Inhibition
Constants (IC₅₀)

| | | Inhibitor |
|---|---|---|
| Transporter | Transfected Cell-line | (Positive control) |
| Pgp | Madin-Darby canine kidney strain II | verapamil |
| | (MDCKII) | |
| BCRP | MDCKII | fumitremorgin C |
| MRP2 | Spodoptera frugiperda (Sf9) ovarian | benzbromarone |
| | cells | |
| BSEP | Sf9 cell membrane vesicles | cyclosporine A |
| NTCP | Chinese Hamster Ovary (CHO) | taurochenodeoxy- |
| | | cholic acid |
| OATP1B1 | CHO | rifampicin |
| OATP1B3 | CHO | rifampicin |

The most potent inhibition observed was for OATP1B1 (range 2 μM to 47 μM). Ambrisentan had no measurable effect on BSEP and NTCP while inhibition was observed for bosentan and macitentan. The most potent inhibition of these transporters was observed for macitentan with IC₅₀ values of 12 and 8 μM for BSEP and NTCP, respectively. Test results are summarized in Table 4.

TABLE 4
Effect of ERAs on Hepatic Uptake and Efflux Transporter

| | IC₅₀ (μM) | | |
|---|---|---|---|
| Transporter | Ambrisentan | Bosentan | Macitentan |
| BSEP | >100 | 54.4 | 12 |
| NTCP | >100 | 36.5 | 8 |
| MRP2 | ~75 | >100 | >100 |
| Pgp | >100 | >100 | 64 ± 15 |
| BCRP | >100 | >100 | 75 ± 37 |
| OATP1B1 | 47.0 ± 21.3 | 5.0 ± 2.0 | 2.0 ± 0.3 |
| OAT1B3 | 44.6 ± 23.8 | 5.2 ± 2.1 | 2.1 ± 0.3 |

Macitentan inhibited hepatic transporters the greatest, while ambrisentan showed little or no effect.

### Example 7

### Clinical Study on Pulmonary Arterial Hypertension (PAH)

Two 12-week, randomized, double-blind, placebo-controlled, multicenter studies were conducted in 393 patients with PAH (WHO Group 1). The two studies were identical in design except for the doses of ambrisentan and the geographic region of the investigational sites. ARIES-1 (Ambrisentan in Pulmonary Arterial Hypertension, Randomized, Double-Blind, Placebo-Controlled, Multicenter, Efficacy Studies) compared once-daily doses of 5 mg and 10 mg ambrisentan to placebo, while ARIES-2 compared once-daily doses of 2.5 mg and 5 mg ambrisentan to placebo. In both studies, ambrisentan or placebo was added to current therapy, which could have included a combination of anticoagulants, diuretics, calcium channel blockers, or digoxin, but not epoprostenol, treprostinil, iloprost, bosentan, or sildenafil. The primary study endpoint was 6-minute walk distance. In addition, clinical worsening, WHO functional class, dyspnea, and SF-36® Health Survey were assessed.

Patients had idiopathic or heritable PAH (64%) or PAH associated with connective tissue diseases (32%), HIV infection (3%), or anorexigen use (1%). There were no patients with PAH associated with congenital heart disease.

Patients had WHO functional class I (2%), II (38%), III (55%), or IV (5%) symptoms at baseline. The mean age of patients was 50 years, 79% of patients were female, and 77% were Caucasian.

Submaximal Exercise Ability:

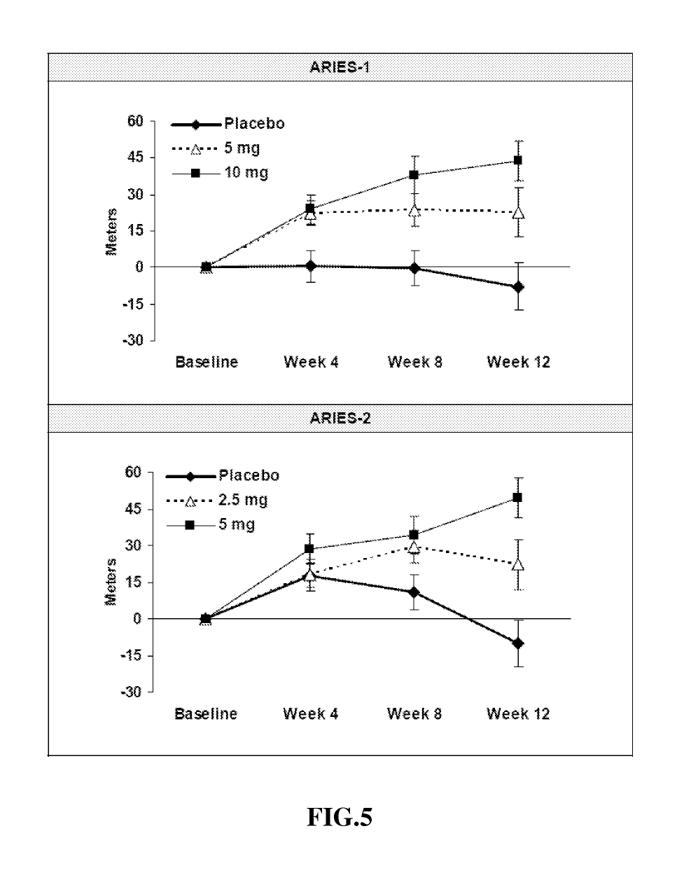

Results of the 6-minute walk distance at 12 weeks for the ARIES-1 and ARIES-2 studies are shown in Table 5 and FIG. 5.

TABLE 5
Changes from Baseline in 6-Minute Walk Distance (meters)

| | ARIES-1 | | | ARIES-2 | | |
|---|---|---|---|---|---|---|
| | Placebo | 5 mg | 10 mg | Placebo | 5 mg | 10 mg |
| | (N = 67) | (N = 67) | (N = 67) | (N = 65) | (N = 64) | (N = 63) |
| Baseline | 342 ± 73 | 340 ± 77 | 342 ± 78 | 343 ± 86 | 347 ± 84 | 355 ± 84 |
| Mean change from | −8 ± 79 | 23 ± 83 | 44 ± 63 | −10 ± 94 | 22 ± 83 | 49 ± 75 |
| baseline | | | | | | |
| Placebo-adjusted mean | — | 31 | 51 | — | 32 | 59 |
| change from baseline | | | | | | |
| Placebo-adjusted | — | 27 | 39 | — | 30 | 45 |
| median change from | | | | | | |
| baseline | | | | | | |
| p-value¹ | — | 0.008 | <0.001 | — | 0.022 | <0.001 |

Mean ± standard deviation
¹p-values are Wilcoxon rank sum test comparisons of ambrisentan to placebo at Week 12 stratified by idiopathic or heritable PAH and non-idiopathic, non-heritable PAH patients.

In both studies, treatment with ambrisentan resulted in a significant improvement in 6-minute walk distance for each dose of ambrisentan and the improvements increased with dose. An increase in 6-minute walk distance was observed after 4 weeks of treatment with ambrisentan, with a dose-response observed after 12 weeks of treatment. Improvements in walk distance with ambrisentan were smaller for elderly patients (age≧65) than younger patients and for patients with secondary PAH than for patients with idiopathic or heritable PAH. The results of such subgroup analyses must be interpreted cautiously. The effects of ambrisentan on walk distances at trough drug levels are not known. Because only once daily dosing was studied in the clinical trials, the efficacy and safety of more frequent dosing regimens for ambrisentan are not known. If exercise ability is not sustained throughout the day in a patient, consider other PAH treatments that have been studied with more frequent dosing regimens.

Clinical Worsening:

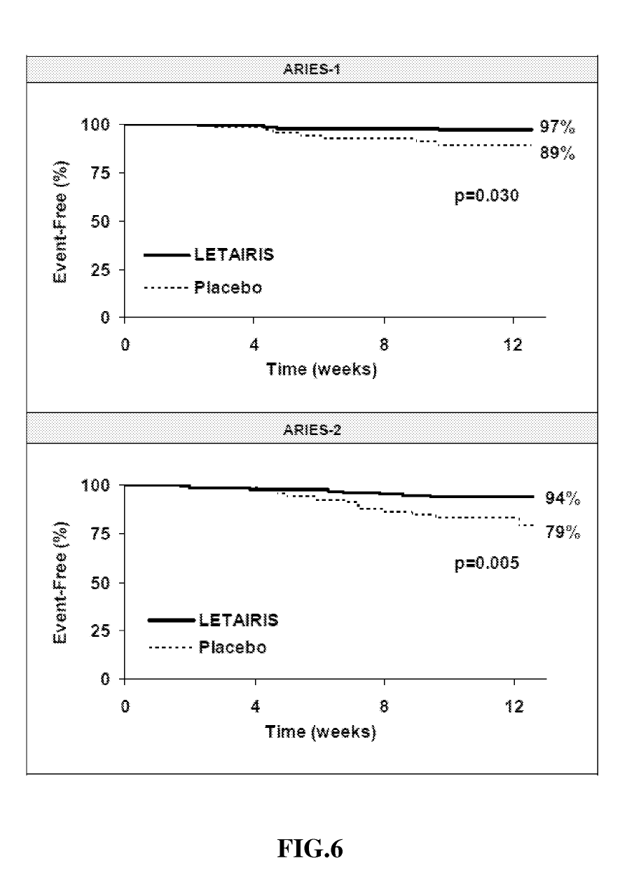

Time to clinical worsening of PAH was defined as the first occurrence of death, lung transplantation, hospitalization for PAH, atrial septostomy, study withdrawal due to the addition of other PAH therapeutic agents or study withdrawal due to early escape. Early escape was defined as meeting two or more of the following criteria: a 20% decrease in the 6-minute walk distance; an increase in WHO functional class; worsening right ventricular failure; rapidly progressing cardiogenic, hepatic, or renal failure; or refractory systolic hypotension. The clinical worsening events during the 12-week treatment period of the ambrisentan clinical trials are shown in Table 6 and FIG. 6.

TABLE 6
Time to Clinical Worsening

| | ARIES-1 | | ARIES-2 | |
|---|---|---|---|---|
| | Placebo | ambrisentan | Placebo | ambrisentan |
| | (N = 67) | (N = 134) | (N = 65) | (N = 127) |
| Clinical worsening, | 7 (10%) | 4 (3%) | 13 (22%) | 8 (6%) |
| no. (%) | | | | |
| Hazard ratio | — | 0.28 | — | 0.30 |
| p-value, Fisher | — | 0.044 | — | 0.006 |
| exact test | | | | |
| p-value, Log-rank | — | 0.030 | — | 0.005 |
| test | | | | |

Intention-to-treat population
Note:
Patients may have had more than one reason for clinical worsening.
Nominal p-values

There was a significant delay in the time to clinical worsening for patients receiving ambrisentan compared to placebo. Results in subgroups such as the elderly were also favorable.

Long-Term Treatment of PAH:

In long-term follow-up of patients who were treated with ambrisentan (2.5 mg, 5 mg, or 10 mg once daily) in the two pivotal studies and their open-label extension (N=383), Kaplan-Meier estimates of survival at 1, 2, and 3 years were 93%, 85%, and 79%, respectively. Of the patients who remained on ambrisentan for up to 3 years, the majority received no other treatment for PAH. These uncontrolled observations do not allow comparison with a group not given ambrisentan and cannot be used to determine the long-term effect of ambrisentan on mortality.

Adverse Effects in Idiopathic Pulmonary Fibrosis (IPF):

A randomized controlled study in patients with IPF, with or without pulmonary hypertension (WHO Group 3), compared ambrisentan (n=329) to placebo (n=163). The study was terminated after 34 weeks for lack of efficacy, and was found to demonstrate a greater risk of disease progression or death on ambrisentan. More patients taking ambrisentan died (8% vs. 4%), had a respiratory hospitalization (13% vs. 6%), and had a decrease in FVC/DLCO (17% vs. 12%).

The words “comprise”, “comprises”, and “comprising” are to be interpreted inclusively rather than exclusively.

What is claimed is:

1. A method of treating pulmonary hypertension in a patient in need thereof, said method comprising:
administering a therapeutically effective amount of ambrisentan to the patient with pulmonary arterial hypertension;
wherein the patient has been determined not to have idiopathic pulmonary fibrosis.

2. The method of claim 1, wherein the subject is administered ambrisentan to improve exercise ability and delay clinical worsening without causing treatment-limiting liver injury.

3. The method of claim 1, wherein the pulmonary hypertension comprises left-sided atrial or ventricular heart disease or left-sided valvular heart disease.

4. The method of claim 1, wherein the pulmonary hypertension is associated with one or more of chronic obstructive pulmonary disease (COPD), sleep-disordered breathing, an alveolar hypoventilation disorder, chronic exposure to high altitude, a developmental abnormality, thromboembolic obstruction of proximal and/or distal pulmonary arteries, a non-thrombotic pulmonary embolism, sarcoidosis, histiocytosis X, lymphangiomatosis or compression of pulmonary vessels.

5. The method of claim 1, wherein the pulmonary hypertension is pulmonary arterial hypertension (PAH).

6. The method of claim 5, wherein the PAH is associated with a connective tissue disease, HIV infection, portal hypertension, a congenital heart disease, schistosomiasis or chronic hemolytic anemia.

7. The method of claim 5, wherein the PAH is associated with one or more of (a) a congenital heart defect, (b) portal hypertension, (c) use of a drug or toxin other than an anorexigen, (d) thyroid disorder, (e) glycogen storage disease, (f) Gaucher disease, (g) hereditary hemorrhagic telangiectasia, (h) hemoglobinopathy, (i) myeloproliferative disorder, (j) splenectomy, (k) pulmonary veno-occlusive disease and (l) pulmonary capillary hemangiomatosis.

8. The method of claim 1, wherein the subject experiences at least one of
(a) adjustment of one or more hemodynamic parameters indicative of improvement of the pulmonary hypertension condition towards a more normal level versus baseline;
(b) increase in exercise capacity versus baseline;
(c) lowering of Borg dyspnea index (BDI) versus baseline;
(d) improvement of one or more quality of life parameters versus baseline;
(e) movement to a lower WHO functional class; and
(f) a reduction in plasma natriuretic peptide levels versus baseline.

9. The method of claim 1, wherein the daily dose of ambrisentan is about 2.5 mg.

10. The method of claim 1, wherein the daily dose of ambrisentan is about 5 mg.

11. The method of claim 10, wherein the daily dose of ambrisentan is increased from about 5 mg to about 10 mg if the about 5 mg ambrisentan daily dose is tolerated by the subject.

12. The method of claim 1, wherein the method consists essentially of:
administering a therapeutically effective amount of ambrisentan to the patient with pulmonary arterial hypertension;
wherein the patient has been determined not to have idiopathic pulmonary fibrosis.

13. The method of claim 1, wherein the ambrisentan is administered in combination therapy with a second active agent effective for treatment of the pulmonary hypertension condition or a condition related thereto, and
wherein the second active agent comprises at least one drug selected from the group consisting of prostanoid, a phosphodiesterase (PDE) inhibitor, a guanylate cyclase activator, a calcium channel blocker, a diuretic, an anticoagulant, oxygen and a combination thereof.

14. The method of claim 13, wherein the PDE5 inhibitor is tadalafil.

15. The method of claim 14, wherein the weight ratio of ambrisentan and the PDE5 inhibitor is in a range from about 1:1.5 to about 1:10.

16. The method of claim 13, wherein the guanylate cyclase activator is riociguat.

17. A method of treating pulmonary arterial hypertension in a patient in need thereof, comprising:
administering a therapeutically effective amount of ambrisentan to the patient with pulmonary arterial hypertension;
wherein the patient has been determined not to have idiopathic pulmonary fibrosis, and
wherein the method is carried out without drug labeling instruction to monitor one or more biomarkers of liver function during ambrisentan treatment.

18. The method of claim 17, wherein the one or more biomarkers include liver aminotransferase or bilirubin.

19. The method of claim 17, wherein the method is carried out without mandatory drug labeling instruction imposed by a regulatory agency to monitor the one or more biomarkers of liver function during ambrisentan treatment.

20. The method of claim 17, wherein the method is carried out without drug labeling instruction to monitor the one or more biomarkers of liver function prior to and during ambrisentan treatment.

21. The method of claim 17, wherein the method is carried out without drug labeling instruction to measure the one or more biomarkers of liver function monthly during ambrisentan treatment.

22. The method of claim 17, wherein the subject has exhibited a grade 1 abnormality in one or more biomarkers of liver function.

23. The method of claim 17, wherein the subject in need thereof has exhibited a grade 2, 3 or 4 abnormality in one or more biomarkers of liver function.

24. The method of claim 17, wherein the ambrisentan is administered in combination therapy with a PDE5 inhibitor effective for treatment of the pulmonary hypertension condition or a condition related thereto.

25. The method of claim 24, wherein the PDE5 inhibitor is tadalafil, and the weight ratio of ambrisentan and tadalafil is in a range from about 1:1.5 to about 1:10.

26. The method of claim 17, wherein the method consists essentially of:
administering a therapeutically effective amount of ambrisentan to the patient with pulmonary arterial hypertension;
wherein the patient has been determined not to have idiopathic pulmonary fibrosis, and
wherein the method is carried out without drug labeling instruction to monitor one or more biomarkers of liver function during ambrisentan treatment.

27. A method of treating pulmonary arterial hypertension in a patient in need thereof, comprising:
diagnosing a patient with pulmonary arterial hypertension;
screening the patient for idiopathic pulmonary fibrosis;
determining that the patient does not have idiopathic pulmonary fibrosis; and
administering a therapeutically effective amount of ambrisentan to the patient with pulmonary arterial hypertension.

28. The method of claim 27, wherein the screening is carried out by performing a diagnostic test on the patient.

29. The method of claim 28, wherein the diagnostic test selected from the group consisting of an imaging technique, lung biopsy, a pulmonary function test, chest X-Ray, CT scan, echocardiogram, blood test, and spirometry.
